(19) United States
(12) Patent Application Publication
(10) Pub. No.: US 2026/0215853 A1
(43) Pub. Date: Jul. 30, 2026

(54) System and Method for Selecting the Proper Cardiac Assist Device for Achieving Improved Strain Profiles When Supporting the Failing or Arrested Heart

(71) Applicant: Lifebridge Technologies LLC, Dayton, OH (US)

(72) Inventor: Mark P. Anstadt, Kettering, OH (US)

(21) Appl. No.: 19/574,284

(22) Filed: Mar. 21, 2026

Related U.S. Application Data
(63) Continuation-in-part of application No. 17/931,853, filed on Sep. 13, 2022, now abandoned.

(51) Int. Cl. A61B 34/10; A61B 5/00

(52) U.S. Cl. CPC A61B 34/10; A61B 5/4851; A61B 2034/107; A61B 2034/108; A61B 2562/0261

(57) ABSTRACT

A methodology for selecting a cardiac assist device and testing that device in vivo. The heart is scanned to obtain dysfunctional heart scan data. A selection of cardiac assist devices and/or elastomeric parts of cardiac assist devices is provided. The dysfunctional heart scan data is compared to functional heart scan data. The differences between the dysfunctional heart scan data and the functional scan data is used to predict which of the cardiac assist devices from the selection available would best minimize the initial differences. An initial cardiac assist device is chosen from the selection. The initial cardiac assist device is positioned in functional contact with the dysfunctional heart. The assisted dysfunctional heart is scanned to obtain updated differences between the dysfunctional heart scan data and the functional scan data. The updated differences are compared to the initial differences to determine if the initial cardiac assist device requires adjustment or replacement.

### RELATED APPLICATIONS

This application is a continuation-in-part of U.S. patent application Ser. No. 17/931,853, filed Sep. 13, 2022.

### BACKGROUND OF THE INVENTION

### 1. Field Of The Invention

In general, the present invention relates to controlling effective functioning of a cardiac assist device coupled to a pneumatic drive system, wherein the cardiac assist device is configured to mechanically support an arrested, failing, or malfunctioning heart. The present invention also relates to the methodology of selecting a cardiac assist device that embodies the properties needed to correct the specific deficiencies of an individual with a dysfunctional heart. More particularly, the present invention relates to systems and methods of determining the forces that need to be applied to a dysfunctional heart to improve the pumping function of the dysfunctional heart and select a cardiac assist device that will provide the needed forces to the dysfunctional heart.

### 2. Prior Art Description

A variety of cardiac assist devices function by imparting external forces to the outer surfaces of an arrested or failing heart from outside the heart. This is done in an effort to eventually restore the physiologic function of the heart. A number of terms have been used to describe these devices and their methods of functioning. The terms include, but are not limited to, direct cardiac compression, cardiac actuation, cardiac massage, mechanical cardiac compression device, and mechanical cardiac massage to name a few. The distinction between the variety of devices used to mechanically pump the heart can be made in such devices with respect to shape, material construct of components acting on the heart, and means for powering their function. Most of these cardiac assist devices focus on aiding at least some component of the heart's pump function. These cardiac assist devices are somewhat similar to each other in that all of them exert mechanical forces onto the heart's surface. However, they vary in their functionalities that involve either primarily compressing the heart and thereby aiding more in systolic pump function (i.e., the process of emptying the heart), or, primarily expanding the heart and thereby aiding more in diastolic pump function (i.e., the process of filling the heart for the next contraction). Some methods provide both diastolic pump function and systolic pump function. The present invention addresses a need for a versatile device that can work in conjunction with a drive system based on an estimate/measurement of the devices strain characteristics and the resulting target systolic and diastolic strain profiles of the heart being acted upon by the cardiac assist device.

Cardiac assist devices are often used to restore or maintain blood flow to vital organs that have become impaired by severe cardiac dysfunction. Many cardiac assist devices use elastomeric material that can expand and contract to create forces that are applied to the heart. There are a variety of elastomeric materials that have been approved for use as part of cardiac assist devices. Each of these elastomeric materials have various characteristics that can be selectively adjusted by varying formulations and/or changing manufacturing variables, such as curing temperature. The characteristics that can be altered include hardness, tensile strength, tensile modulus, elongation, resilience, compression set, tear resistance, abrasion resistance, and specific gravity. Each of these characteristics effects how the elastomeric material reacts to applied forces.

In cardiac assist devices, elastomeric materials are used for a variety of purposes. For example, an elastomeric cuff may be placed around the ventricles of the heart to assist the heart in contracting. In each application, there are optimal characteristics that the elastomeric material should embody. The physical characteristics of elastomeric material are best measured with reference to strain. Strain is the measure of how much an object deforms or changes shape, such as stretching or compressing, in response to applied forces. Mathematically, strain is defined as the ratio of deformation to the original dimension. Cardiac assist devices that contain elastomeric materials are designed to embody certain strain dynamics that are created by the elastomeric material. The strain dynamics quantify how much the elastomeric material stretches, compresses and/or twists as the cardiac assist device operates. The strain dynamics selected for a cardiac assist device depends both upon the application and on the unique anatomy and/or pathologic conditions of the heart. For example, two patients of similar demographics can have different stages of heart disease. Accordingly, different forces must be applied to the heart according to its anatomic characteristics and the underlying dysfunction of the heart. For example, different degrees of heart failure require different degrees of support. Another example is the heart's underlying muscle thickness. A dilated heart typically exhibits a thin dilated muscle (dilated cardiomyopathy) whereas a heart diseased by long standing high blood pressure has a thick muscle (hypertrophic cardiomyopathy). This different heart conditions and/or characteristics require the use of different elastomeric materials and/or different cardiac assist devices to properly assist the dysfunctional heart with proper strain dynamics. In turn, the ideal pump characteristics of the heart are exhibited by a set of strain/strain rate profiles. Therefore, the optimal heart pump strain dynamics can be extrapolated from the resulting heart strain and/or strain rate profiles when the device acts on the heart.

Currently, the strain profile embodies by elastomeric materials in cardiac assist devices are poorly understood and underdeveloped. It is difficult to predict or otherwise model what elastomeric materials will best produce the forces that must be created by a cardiac assist device. The best predictor of the forces that will be produced by a cardiac assist device is target profiles for strains/strain rates (e.g., left ventricular (LV) strain or LV strain rate) measured across the patient's body or from within the blood circulation. Each of the target profiles can be for systolic support, diastolic support, or both systolic and diastolic support.

Notably, the strain/strain rate profiles provide unique means for segregating the function imposed externally by the pump from the native function that the heart itself. When the pump is off, strain or strain rate profiles represent the heart's native function, when the heart is not completely arrested. On the other hand, when the heart is arrested, i.e., not beating, strain/strain rate profiles strictly represent the function imparted by the pump. Other estimations of how the device act on the heart are less than ideal. Arriving at an ideal device to achieve target profiles may require altering the selected elastomeric construct of the device when it does not perform optimally.

In the prior art, there are cardiac assist devices that embody elastomeric materials and contain strain gauges. These constructs enable a surgical team to directly measure strains that are being experienced by the cardiac assist device. Such prior art is exemplified by U.S. patent application Ser. No. 17/825,343 to Anstadt. Such devices provide data about the forces being experienced by the cardiac assist device. The data, however, does not always accurately reflect the forces being applied to the body by the cardiac assist device. Often, the data is limited to the position and orientation of the strain gauges in the construct.

Furthermore, such devices require wire leads. The wire leads apply forces to the construct that also affects the data. Furthermore, the use of wire leads prohibits the use of MRI imaging and other metal sensitive imaging techniques.

A need therefore exists for an improved system and method of determining the proper characteristics of a cardiac assist device to be used for a specific in vivo application then selecting the best construction of a cardiac assist device to suit the need. This need is met by the present invention as described and claimed below.

### SUMMARY OF THE INVENTION

The present invention is a methodology for selecting a cardiac assist device and testing that cardiac assist device in vivo. To select a cardiac assist device for a dysfunctional heart, the dysfunctional heart is first scanned to obtain dysfunctional heart scan data.

As noted in the background, a heart's pump function can be separated into systolic pump function (i.e., the process of ejecting blood from the heart) and diastolic pump function (the process of filling the heart for the next contraction). These two components are distinct and cardiac assist devices can affect either one or both although the intent of the cardiac assist device may be more for one or the other. The intention of acting on just one of the systolic and diastolic component of pump function can lead to a secondary adverse effect on the other. For example, merely compressing the heart with a mechanical device can augment filling. However, absent a concerted and direct beneficial action toward diastolic function, such a device can actually impair diastolic pump function. Therefore, the solutions disclosed in this patent application are directed towards the proper independent control of both systolic and/or proper diastolic pump function. This ability is particularly important because conditions of cardiac arrest or heart failure exhibit both filling (diastolic function) and emptying (systolic function) dysfunction. A selection of cardiac assist devices and/or elastomeric parts of cardiac assist devices are provided. The dysfunctional heart scan data is compared to functional heart scan data from a healthy heart. The initial differences between the dysfunctional heart scan data and the functional scan data is used to predict which of the cardiac assist devices from the selection available would best minimize the initial differences.

The difference between the dysfunctional heart and an ideal functional state can be determined by measures of strain in a heart which can be acquired by a variety of relevant sensors or gauges within a cardiac assist device or within the vascular system or on the surface of the patient's body. A typical example would be an echocardiogram probe placed at relevant locations. Either strain or strain rate profiles can be measured to provide the state of the heart's dysfunction and determine the ideal corrected target state during device support. The parameters of strain/strain-rate-based heart function can be achieved by an echo probes placed on the surface of the body or acquired if integrated within the device itself such as piezo-electric strain sensor is integrated within the pump to measure change of length of the device. An additional advantage of the strain/strain-rate is that the measure of stain can also be applied to the estimation/measurement of device strain characteristics while acting on the heart.

An initial cardiac assist device is chosen from the selection of cardiac assist devices. The chosen device is the one predicted to best minimize the initial differences. The initial cardiac assist device is positioned in contact with the dysfunctional heart in vivo. The initial cardiac assist device is operated to affect the dysfunctional heart.

The assisted dysfunctional heart is scanned to obtain updated differences between the dysfunctional heart scan data and the functional scan data. The updated differences are compared to the initial differences to determine if the initial cardiac assist device requires adjustment or replacement. If a better cardiac assist device is needed, the cardiac assist device is either removed and replaced or removed and refitted with more appropriate elastomeric parts.

### BRIEF DESCRIPTION OF THE DRAWINGS

For a better understanding of the present invention, reference is made to the following description of an exemplary embodiment thereof, considered in conjunction with the accompanying drawings, in which:

FIG. 1 is schematic of an exemplary embodiment of a scanning system for scanning a heart being acted upon by a cardiac assist device;

FIG. 2 is an enlarged view of the exemplary cardiac assist device;

FIG. 3 shows a strain curve between two exemplary points on an elastomeric construct within the cardiac assist device; and

FIG. 4 is a block diagram outlining the method of operation for the present invention methodology.

### DETAILED DESCRIPTION OF THE DRAWINGS

Although the present invention methodology can be embodied in many ways, only some exemplary methods are illustrated and described. The exemplary embodiments are being shown for the purposes of explanation and description. The exemplary embodiments are selected in order to set forth some of the best modes contemplated for the invention. The illustrated embodiments, however, are merely exemplary and should not be considered as limitations when interpreting the scope of the appended claims.

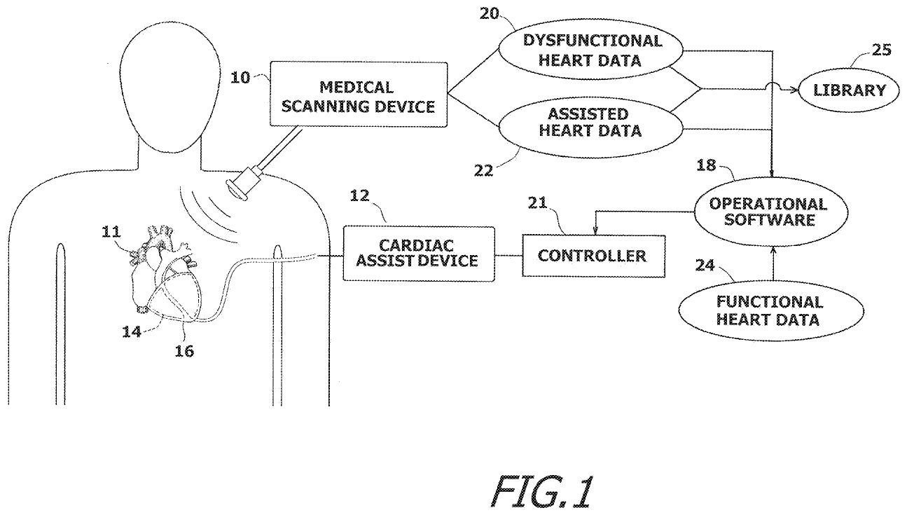

Referring to FIG. 1, a medical imaging system 10 is used to image a dysfunctional heart 11 and any cardiac assist device 12 that is attempting to assist the dysfunctional heart 11. The medical imaging system 10 can be an x-ray imaging system, a CT imaging system, an MRI imaging system, an ultrasound imaging system, a PET imaging system, an electromagnetic imaging system, a fluoroscope imaging system, a spectroscopy imaging system, or an optic imaging system, such as an optical coherence tomography system.

Initially, the imaging system 10 is used to image the dysfunctional heart 11. This produces dysfunction heart scan data 20. As will be explained, after a cardiac assist device 12 is placed in functional contact with the dysfunctional heart 11, the imaging system 10 images the cardiac assist device 12 in vivo as it acts upon the dysfunctional heart 11. This creates assisted heart scan data 22 that is determinative of how the cardiac assist device 12 reacts to forces applied to the dysfunctional heart 11 and how the dysfunctional heart 11 reacts to forces applied by the cardiac assist device 12. The assisted heart scan data 22 is indicative of the dynamic strains experienced by the tissue within the dysfunctional heart 11.

Both the dysfunctional heart scan data 20 and the assisted heart scan data 22 are saved in a reference library 25 along with the anatomical characteristics of the dysfunctional heart and the physical characteristics of the cardiac assist device 12. For example, the dysfunctional heart that is scanned to produce the dysfunctional heart scan data 20 may have a given volume, sphericity, length, max diameter, ejection fraction, and cardiac output. The dysfunctional heart scan data 20 contains the various strain profiles that are associated with different sections of the heart at various points in the cardiac cycle. These values are searchable variables in the reference library 25. Likewise, the type, model, dimensions, and strain characteristics of the cardiac assist device 12 are saved as searchable variables in the reference library 25.

When the cardiac assist device 12 acts upon the dysfunctional heart 11, the medical scanning device 10 measures the assisted heart scan data 22. This data is also saved in the reference library 25. The result is that data is collected that directly associates the variables of cardiac assist devices 12 to improved heart function in dysfunctional hearts of different types. Once enough data is collected to perform a reliable statistical analysis, the reference library can be utilized to initially select the proper cardiac assist device 12 for various types of dysfunctional hearts 11.

The cardiac assist device 12 can be any implanted or inserted device that contains elastomeric components 14 and is intended to apply forces to, and/or absorb forces from, the dysfunctional heart 11. The illustrated example shows a heart pump cup 16.

The cardiac assist device 12 is set in vivo during a surgical procedure. Once set, the heart 11 is imaged using the medical imaging system 10. This produces the assisted heart scan data 22. Thee heart scan data 22 can contain heart strain information and/or heart sphericity information. The assisted heart scan data 22 indicates how the heart 11 is reacting to the forces that are being applied by the cardiac assist device 12. The assisted heart scan data 22 is analyzed by a computer controller 21 that is running appropriate data analysis software 18. By analyzing the assisted heart scan data 22, it can be determined if forces being applied or absorbed in various areas of the cardiac assist device 12 are too great or too small, given the composition and shape of the elastomeric components 14 in use. In this manner, changes in the cardiac assist device 12 can be suggested in real time. The cardiac assist device 12 can then be replaced or reconfigured with elastomeric components 14 that are softer, harder, thinner, thicker, or otherwise formed to better provide the lacking strain characteristics that are indicated by the dysfunctional heart data 20.

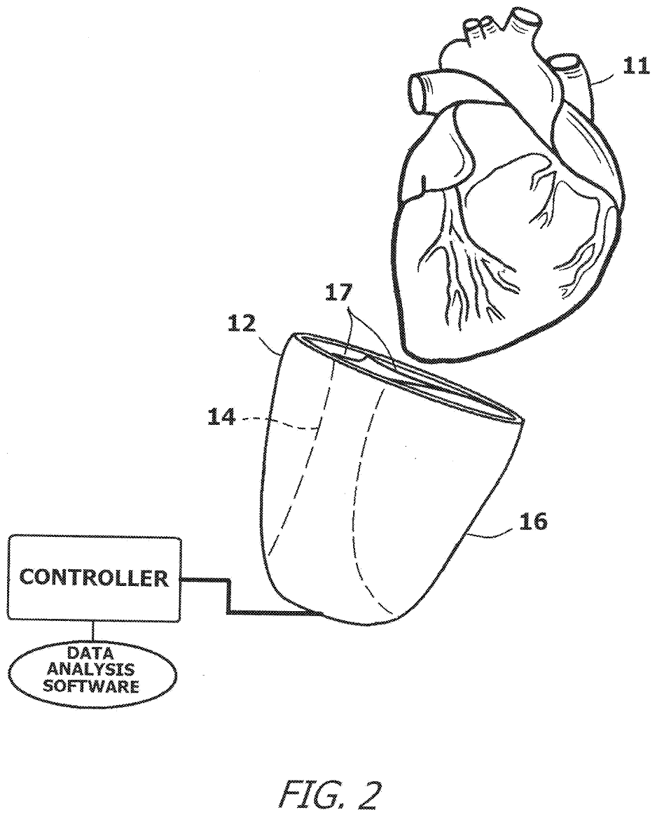

Most cardiac assist devices 12 apply active forces to the dysfunctional heart 11 in addition to the passive forces caused by the elastic materials in the elastomeric components 14. For example, the cardiac assist device 12 may expand or contract upon the application of pneumatic, hydraulic, or electromechanical forces. Referring to FIG. 1 in conjunction with FIG. 2, it will be understood that the cardiac assist device 12 must apply very specific forces to the dysfunctional heart 11 at specific times. The exemplary cardiac assist device 12 is a heart pump cup 16 with internal elastomeric bladders 17 that can selectively expand and/or contract to apply forces to the dysfunctional heart 11. In this manner, the cardiac assist device 12 can help the dysfunctional heart 11 maintain variable pumping function. The forces that are applied to the dysfunctional heart 11 by the cardiac assist device 12 are partially due to the elastomeric components 14. In the illustrated embodiment, the bladders 17 serves as the elastomeric components 14. The bladders 17 can be permanently affixed to the heart pump cup 16 or the bladders 17 can be selectively replaceable. Once in place, the elastomeric bladders 17 will expand and contract as a function of external forces being generated using hydraulic or pneumatic means. In such a system, the strain characteristics embodied by the cardiac assist device 12 are highly dependent upon the characteristics of the elastomeric material used in forming the bladders 17 and the thicknesses of the elastomeric material at different points along the bladder 17.

The medical imaging system 10 is capable of detecting and measuring changes in shape, size, and strain profile of the dysfunctional heart 11 and the cardiac assist device 12 acts upon the dysfunctional heart 11. The displacement of the surfaces of the dysfunctional heart 11 over time creates strain profiles for these surfaces.

Many elastomeric materials in cardiac assist devices have strain dynamics that can be interrogated using the listed imaging modalities. Additionally, the presence of the elastomeric components 14 within or against the dysfunctional heart 11 can enhance the ability to assess the various strains as well as the relationship between the material strain dynamics and tissue strain dynamics.

In order to fabricate a cardiac assist device 12 that meets the strain and/or sphericity characteristics of a healthy functional heart, the strain and/or sphericity characteristics for the healthy heart must first be determined to obtain healthy heart scan data 24. This can be accomplished using statistical functional heart data obtained from historical medical imaging. Furthermore, functional heart scan data 24 can be obtained using mathematical modeling or by physically testing anatomical models.

Using the medical imaging equipment 10, the dysfunctional heart 11 of a patient can be scanned at one or more times prior to surgery. From the medical imaging scans, tissue deformations can be detected and measured. The deformations in total can be considered an amalgamation of dozens, hundreds, or thousands of point-to-point deformations in the heart tissue.

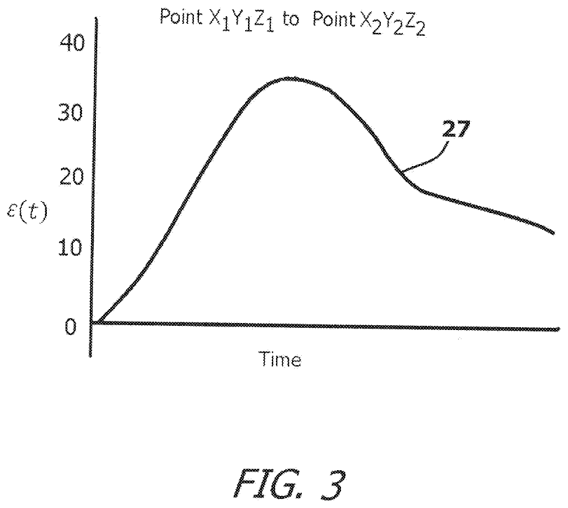

Referring to FIG. 3 in conjunction with FIG. 1 and FIG. 2, it can be seen that starting at any one point in the heart tissue at coordinates X₁Y₁Z₁ to any second point in the heart tissue at coordinates X₂Y₂Z₂, there is a deformation profile 27 that occurs over time. The deformation profile 27 is a graphical representation of the dysfunctional heart scan data 20. Once the cardiac assist device 12 acts upon the dysfunctional heart 11, the assisted heart scan data 22 changes. The coordinates throughout the heart tissue are supplemented with, the forces provided by and absorbed by the cardiac assist device 12.

The dynamic strain profile for a healthy heart is known or can be readily calculated measuring a few heart dimensions that are obtainable from the medical imaging of the heart. The method of calculating a strain profile for a heart is described in detail in U.S. patent application Ser. No. 17/825,343, the disclosure of which is herein incorporated by reference.

In the application of a cardiac assist device 12 to a dysfunctional heart 11, the cardiac assist device 12 should apply forces to the heart 11 that cause the measured dysfunctional heart scan data 20 to approach the healthy heart scan data 24 that is calculated for a healthy heart. Thus, the optimal characteristics for the forces applied by the cardiac assist device 12 correspond to the differences between the healthy heart scan data 24 and the dysfunctional heart scan data 20.

The forces that need to be applied by the cardiac assist device 12 determine the strain characteristics that need to be embodied by the cardiac assist device 12. Once the strain characteristics to be embodied are believed to be known, the surgical team selects an initial cardiac assist device 12 that matches the calculated characteristics needed to change the dysfunctional heart scan data 20 of the patient's heart into the healthy heart scan data 24 of a functional heart. The cardiac assist device 12 is then set in vivo. Once in place, the heart 11 is imaged using the medical imaging system 10 in order to determine the actual dynamic strain profile of the assisted heart in vivo. Due to the many variables concerning the tissue being effected by the cardiac assist device 12, the actual dynamic strain profile for the heart 11 can vary significantly from the expected strain characteristics. If the measured dynamic strain profile shows that the cardiac assist device 12, in part or whole, w applies forces other than those required, then the cardiac assist device 12 is removed. The cardiac assist device 12 can be replaced or altered. If altered, some or all of the elastomeric components 14 can be replaced with components having elastomeric components 14 that are thicker, thinner, more pliant, less pliant, or otherwise altered to a different formulation. Likewise, if the elastomeric components 14 deform less than is desired, then the elastomeric component 14 can be changed for one of a different formulation. Changes in formulations are generally pursued if many of the areas within the cardiac assist device 12 turn out to be too soft or too stiff. Alternatively, if many areas of the elastomeric component 14 are too flexible and/or soft and many areas too elastic or stiff, then the elastomeric component 14 can be changed for one that us thinned and thickened as needed without change to the formulation of the material used in the formation of the elastomeric component 14.

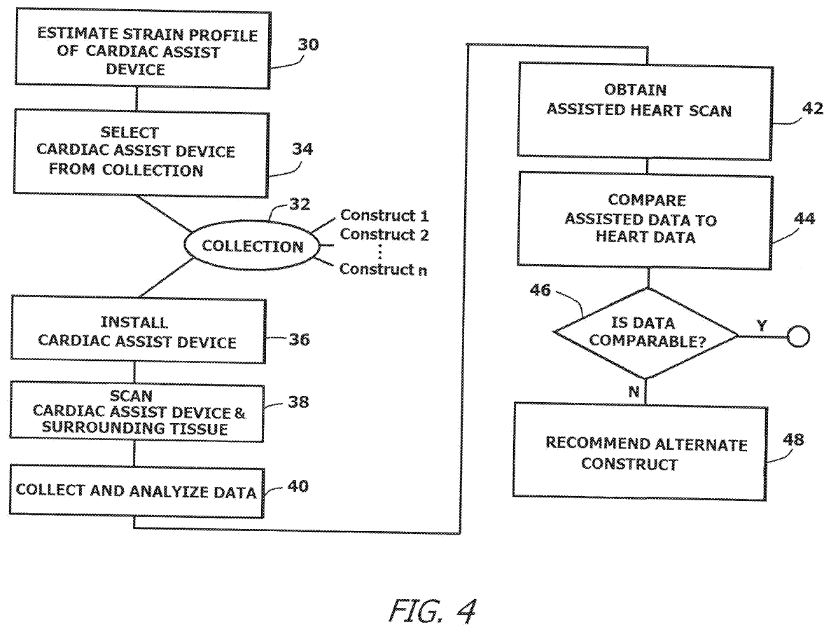

Referring to FIG. 4 in conjunction with FIG. 1, FIG. 2, and FIG. 3, it will be understood that the first step in determining what elastomeric material 14 and dimensions to use in a cardiac assist device 12, is to estimate the forces that must be supplied to and/or resisted by the cardiac assist device 12. As stated, this can be accomplished by comparing the dysfunctional heart scan data 20 to the healthy heart scan data 24. See Block 30.

The general shape of the cardiac assist device 12 is known and is dictated by its purpose. For example, a heart cup must fit around the ventricles of a heart. Furthermore, the variations in strain characteristics typically fall within a confined range for a given application. A collection 32 of cardiac assist devices 12 and/or elastomeric components 14 for cardiac assist devices 12 are provided that together span the possible range. Given the general shape of the cardiac assist device 12 and an estimation of the strain characteristics that must be present. The data stored in the reference library 25 us searched select an initial cardiac assist device 12 is selected from the collection 32. See Block 34. The selected cardiac assist device 12 is an educated guess based upon statistical analysis of the data in the reference library 25 and the experiences of the surgical team.

The selected cardiac assist device 12 is then surgically installed. See Block 36. Once installed, the heart tissue effected by the cardiac assist device 12 is actively scanned using traditional medical scanning systems. See Block 38. The data collected from the scan is processed using the data analysis software 18. See Block 40. The data analysis software 18 measures the actual deformations in the heart tissue. Knowing what cardiac assist device 12 was installed as well as the properties and dimensions of the selected cardiac assist device 12, the deformations experienced by the heart tissue can be readily converted into assisted heart scan data 22. See Block 42. The assisted heart scan data 22 is then compared to the healthy heart scan data 24 calculated by the surgical team prior to the surgical procedure. See Block 44. If the assisted heart scan data 22 is close to the healthy heart scan data 24, then a successful selection was made and the surgical procedure can be completed. See Block 46. However, if the assisted heart scan data 22 differs significantly from the healthy heart scan data 24, then it can readily be determined that the selected initial cardiac assist device 12 lacks the proper elastomeric components 14. The data analysis software 18 then recommends another of the cardiac assist devices 12 and/or elastomeric components 14 from the collection 32, that may produce better results. Alternatively, the data analysis software 18 can recommend changes to a particular elastic component 14 of a cardiac assist device 12 to make that cardiac assist device 12 better to meet the needs of the dysfunctional heart 11. See Block 48.

If the data analysis software 18 indicates that an alternate cardiac assist device 12 is more suitable than the one initially selected, the substitution is preferably made during the same surgical or insertion procedure. If the cardiac assist device 12 is to be replaced, the data analysis software 18 selects a more appropriate cardiac assist device 12 from the collection 32. For example, if the cardiac assist device 12 is a cup with expandable bladders and it is determined that a bladder is too stiff, the data analysis software 18 will indicate that an alternative cardiac assist device that is more elastic be used.

Alternatively, if the data analysis software 18 indicates that one of the internal bladders 17 are too stiff or too elastic, then the data analysis software 18 can recommend a replacement for that bladder. The cardiac assist device 12 can be removed, the bladder 17 quickly replaced and the cardiac assist device 12 reinstalled in the operating room. Furthermore, data can be collected for the selection for the configuration of the cardiac assist device 12 for use in predicting the proper cardiac assist device 12 for subsequent procedures.

It will be understood that the embodiments of the present invention that are illustrated and described are merely exemplary and that a person skilled in the art can make many variations to those embodiments. All such embodiments are intended to be included within the scope of the present invention as defined by the claims.

What is claimed is:

1. A method of selecting a cardiac assist device and testing said cardiac assist device in vivo, comprising;
scanning a dysfunctional heart to obtain dysfunctional heart scan data;
providing a selection of cardiac assist devices;
comparing said dysfunctional heart scan data to functional heart scan data;
using initial differences between said dysfunctional heart scan data and said functional heart scan data to predict which of said cardiac assist devices from said selection of cardiac assist devices best minimizes said initial differences;
selecting an initial cardiac assist device from said selection of cardiac assist devices that is predicted to best minimize said initial differences;
placing said initial cardiac assist device in contact with said dysfunctional heart in vivo;
operating said initial cardiac assist device in vivo;
scanning said dysfunctional heart to obtain assisted heart scan data; and
comparing said assisted heart scan data to said functional heart scan data to determine if said initial cardiac assist device requires replacement.

2. The method according to claim 1, wherein scanning a dysfunctional heart to obtain dysfunctional heart scan data includes measuring a strain and/or a strain rate in a wall of the dysfunctional heart.

3. The method according to claim 2, wherein said strain and/or said strain rate is used to determine an optimal effect of said cardiac assist device when acting on the dysfunctional heart.

4. The method of claim 3, wherein said strain and/or a strain rate in a wall of the dysfunctional heart produces a set of target profiles that corresponds to one or both of a systolic phase and a diastolic phase of the dysfunctional heart.

5. The method according to claim 1, further including replacing said cardiac assist device with another from said selection should differences between said assisted heart scan data and said functional heart scan data differ beyond a predetermined range.

6. The method according to claim 1, wherein said cardiac assist device has alterable features and said method further includes altering at least one of said features should differences between said assisted heart scan data and said functional scan heart data differ beyond an acceptable range.

7. The method according to claim 1, wherein scanning includes scanning said dysfunctional heart with a medical imaging system selected from a group consisting of X-ray imaging systems, CT imaging systems, MRI imaging systems, ultrasound imaging systems, PET imaging systems, electromagnetic imaging systems, fluoroscopic imaging systems, spectroscopy imaging systems, and optical imaging systems.

8. The method according to claim 1, wherein said cardiac assist device has at least one component made from elastomeric material.

9. A method of selecting a cardiac assist device and testing said cardiac assist device in vivo, comprising;
scanning a dysfunctional heart to obtain dysfunctional heart scan data;
providing a selection of elastomeric components for a cardiac assist device;
comparing said dysfunctional heart scan data to functional heart scan data;
using initial differences between said dysfunctional heart scan data and said functional scan data to predict which of elastomeric components from said selection of elastic components would work best in said cardiac assist device to minimizes said initial differences;
selecting elastomeric components from said selection of elastomeric components that are predicted to best minimize said initial differences and installing said elastic components into said cardiac assist device;
placing said cardiac assist device in contact with said dysfunctional heart in vivo;
operating said cardiac assist device in vivo;
scanning said heart obtain updated differences between said dysfunctional heart scan data and said functional scan data;
comparing said updated differences to said initial differences to determine if said elastomeric components in said cardiac assist device require replacement.

10. The method according to claim 9, further including replacing at least one said elastomeric component with another from said selection should said updated differences between said dysfunctional heart scan data and said functional scan data differ beyond a predetermined range.

11. The method according to claim 10, wherein scanning said cardiac assist device in vivo includes scanning said cardiac assist device with a medical imaging system selected from a group consisting of X-ray imaging systems, CT imaging systems, MRI imaging systems, ultrasound imaging systems, PET imaging systems, electromagnetic imaging systems, fluoroscopic imaging systems, spectroscopy imaging systems, and optical imaging systems.

12. A method of selecting a cardiac assist device, comprising;
measuring a dysfunctional heart to obtain physical characteristics embodied by said dysfunctional heart;
scanning a dysfunctional heart to obtain dysfunctional heart scan data, wherein said physical characteristics and said dysfunctional heart scan data are used to determine a strain profile for said dysfunctional heart;
providing a selection of cardiac assist devices;
selecting an initial cardiac assist device from said selection of cardiac assist devices;
placing said initial cardiac assist device in functional contact with said dysfunctional heart;
scanning said dysfunctional heart while acted upon by said initial cardiac assist device to acquire an assisted cardiac strain profile;
comparing said assisted cardiac strain profile to a standard strain profile of a functional heart to determine if said initial cardiac assist device requires replacement.

13. The method according to claim 12, further including replacing said initial cardiac assist device with another from said selection should said assisted cardiac strain profile differ from said standard strain profile outside of a predetermined range.

14. The method according to claim 12, wherein said cardiac assist device has alterable features and said method further includes altering at least one of said features should said assisted cardiac strain profile differs from said standard strain profile outside an acceptable range.

15. The method according to claim 12, wherein scanning includes scanning said dysfunctional heart with a medical imaging system selected from a group consisting of X-ray imaging systems, CT imaging systems, MRI imaging systems, ultrasound imaging systems, PET imaging systems, electromagnetic imaging systems, fluoroscopic imaging systems, spectroscopy imaging systems, and optical imaging systems.

16. The method according to claim 12, wherein said cardiac assist device has at least one component made from elastomeric material.

17. A method of selecting a cardiac assist device to act upon a heart, comprising:
obtaining patient-specific cardiac strain data;
accessing a library of predefined device strain profiles associated with a selection of cardiac assist devices;
comparing said patient-specific cardiac strain data to said predefined device strain profiles in said library; and
selecting an initial cardiac assist device whose predefined device strain profile best approximates a target myocardial strain profile.

18. The method of claim 17, further including placing said initial cardiac assist device in functional contact with the heart and scanning the heart while acted upon by said initial cardiac assist device to acquire an assisted cardiac strain profile.

19. The method according to claim 18, further including comparing said assisted cardiac strain profile to said target myocardial strain profile to determine if said initial cardiac assist device requires replacement.

20. The method according to claim 19, further including replacing said initial cardiac assist device with another from said selection should said assisted cardiac strain profile differ from said target myocardial strain profile outside of a predetermined range.

21. The method according to claim 19, wherein said cardiac assist device has alterable features and said method further includes altering at least one of said features should said assisted cardiac strain profile differs from said target myocardial strain profile outside an acceptable range.

22. A method of selecting a cardiac assist device, comprising:
obtaining patient-specific cardiac strain data;
accessing a library of device strain profiles associated with cardiac assist devices;
comparing said patient-specific cardiac strain data to said predefined device strain profiles; and
selecting a cardiac assist device having a predefined device strain profile that best approximates a target myocardial strain profile.

23. The method of claim 22, further including placing said initial cardiac assist device in functional contact with a heart and scanning the heart while acted upon by said initial cardiac assist device to acquire an assisted cardiac strain profile.

24. The method according to claim 23, further including comparing said assisted cardiac strain profile to said target myocardial strain profile to determine if said initial cardiac assist device requires replacement.

25. The method according to claim 22, further including replacing said initial cardiac assist device should said assisted cardiac strain profile differ from said target myocardial strain profile outside of a predetermined range.

26. The method according to claim 22, wherein said cardiac assist device has alterable features and said method further includes altering at least one of said features should said assisted cardiac strain profile differs from said target myocardial strain profile outside an acceptable range.
